(19) United States
(12) Patent Application Publication
(10) Pub. No.: US 2012/0130280 A1
(43) Pub. Date: May 24, 2012

(54) LEGS REHABILITATION DEVICE AND LEGS REHABILITATION METHOD USING THE SAME

(75) Inventor: Yang-Soo Lee, Daegu (KR)

(73) Assignee: Industry-Academic Cooperation Foundation, Kyungpook National University, Daegu (KR)

(21) Appl. No.: 13/299,333

(22) Filed: Nov. 17, 2011

(30) Foreign Application Priority Data
Nov. 18, 2010 (KR) 10-2010-0115094

(51) Int. Cl. A61B 5/103

(52) U.S. Cl. 600/587

(57) ABSTRACT

Disclosed is a legs rehabilitation device. The legs rehabilitation device according to an exemplary embodiment of the present invention includes a first sensor that senses a load applied from a support surface of one leg of a rehabilitating patient; a second sensor that senses an angle of the one leg; a third sensor that senses position or motion of the other leg of the rehabilitating patient; and a rehabilitation determination unit for communicating with the first sensor, the second sensor, and the third sensor and determines whether the legs rehabilitation succeeds, based on load information received through the first sensor, angle information received through the second sensor, and position or motion information received through the third sensor.

### CROSS-REFERENCE TO RELATED APPLICATIONS

This application claims priority to and the benefit of Korean Patent Application No. 10-2010-0115094 filed in the Korean Intellectual Property Office on Nov. 18, 2010, the entire contents of which are incorporated herein by reference.

### TECHNICAL FIELD

The present invention relates to a rehabilitation device, and more particularly, to a legs rehabilitation device for improving a function of legs with the degraded weight support capability and balance capability and a legs rehabilitation method using the same.

### BACKGROUND

When a body is paralyzed due to a stroke, a traumatic brain injury, or the like, or muscular strength weakens due to aging or nonuse, the muscle does not show necessary normal strength and nor generate necessary strength. The degradation in the leg function causes patients not to normally support a body weight in the case of standing or walking and walking abnormality. Recently, attempts to allow patients with legs having the degraded function to forcibly use their legs as a treatment are being increased.

However, since patients with legs having the degraded function does cannot freely use move the degraded legs, they do not want to use the legs, such that the function of the legs becomes further degraded.

A need exists for a legs rehabilitation device suitable to normalize the function of the legs.

### SUMMARY

The present invention has been made in an effort to provide a legs rehabilitation device for improving support capability of legs having a degraded function rehabilitation method using the same.

Further, the present invention has been made in an effort to provide a legs rehabilitation device capable of feed-backing a rehabilitation operation to a user in order to improve support capability of legs having the degraded function and a legs rehabilitation method using the same.

An exemplary embodiment of the present invention provides a legs rehabilitation device, including: a first sensor that senses a load applied from a support surface of one leg of a rehabilitating patient; a second sensor that senses an angle of one leg; a third sensor that senses position or motion of the other leg of the rehabilitating patient; and a rehabilitation determination unit that communicates with the first sensor, the second sensor, and the third sensor and determines whether the legs rehabilitation succeeds, based on load information received through the first sensor, angle information received through the second sensor, and position or motion information received through the third sensor.

The rehabilitation determination unit may determine that the rehabilitation fails, when the load information received through the first sensor is below a predetermined reference load or the angle information received through the second sensor is out of a predetermined reference angle range, while the position or motion of the other leg received through the third sensor reaches a target.

The rehabilitation determination unit may determine that the legs rehabilitation fails, when the cursor moving by the position or motion of the other leg received through the third sensor does not reach a target.

The rehabilitation determination unit may determine that the legs rehabilitation succeeds, when the load information received through the first sensor is a predetermined reference load or more and the angle information received through the second sensor is within the predetermined reference angle range, while the cursor moving by the position or motion of the other leg received through the third sensor reaches a target.

The first sensor may be a load sensor that senses the load applied from one leg through the support surface.

The second sensor may be an angle sensor that is attached to one leg of the rehabilitating patient to measure an angle of the leg.

The third sensor may be a sensor that is attached to the other leg of the rehabilitating patient to sense the position or motion of the other leg.

The sensor may be a sensor sensing the position or motion, such as an accelerometer, a gyro sensor, a contact sensor, or the like.

The position sensor may be a touch sensor that receives the positional information by contacting one of a plurality of touch sensors having the predetermined positional information.

The rehabilitation determination unit may provide a game function to the rehabilitating patient, based on the load information received through the first sensor, the angle information received through the second sensor, and the position or motion information received through the third sensor.

The rehabilitation determination unit may include: a sensor interface unit that communicates with the first sensor, the second sensor, and the third sensor; a controller that generates operation success information when the load measured in the first sensor is the reference load or more, while the position or motion information is provided through the sensor interface unit from the third sensor and generates operation failure information when the load measured in the first sensor is below the reference load, while the position or motion information is provided through the sensor interface unit from the third sensor; and an output unit that outputs the operation success information and the operation failure information.

Another exemplary embodiment of the present invention provides a legs rehabilitation device, including: a first sensor that senses a load applied from a support surface of one leg of a rehabilitating patient; a third sensor that senses position or motion of the other leg of the rehabilitating patient; and a rehabilitation determination unit that communicates with the first sensor and the third sensor and determines whether the legs rehabilitation succeeds, based on load information received through the first sensor and the position or motion information received through the third sensor.

The rehabilitation determination unit may determine that the legs rehabilitation fails, when the load information received through the first sensor is below a predetermined reference load, while a cursor moving by the position or motion information of the other leg received through the third sensor reaches a target.

The rehabilitation determination unit may determine that the legs rehabilitation succeeds, when the load information received through the first sensor is the predetermined reference load or more, while the cursor moving by the position or motion information received through the third sensor reaches a target.

The rehabilitation determination unit may include: a sensor interface unit that communicates with the first sensor and the third sensor; a controller that generates operation success information when the load measured in the first sensor is the reference load or more and generates operation failure information when the load measured in the first sensor is below the reference load, while a sensing value of the third sensor is provided through the sensor interface unit; and an output unit that outputs the operation failure information.

Yet another exemplary embodiment of the present invention provides a legs rehabilitation device, including: a first sensor that senses a load applied from a support surface of one leg of a rehabilitating patient; a second sensor that senses an angle of one leg; and a rehabilitation determination unit that communicates with the first sensor and the second sensor and determines whether the legs rehabilitation succeeds, based on load information received through the first sensor and angle information received through the second sensor.

The rehabilitation determination unit may determine that the legs rehabilitation fails, when the load information received through the first sensor is below a predetermined reference load, while the angle information received through the second sensor is within a predetermined angle range.

The rehabilitation determination unit may determine that the legs rehabilitation succeeds, when the load information received through the first sensor is a predetermined reference load or more, while the angle information received through the second sensor is within a predetermined angle range.

The rehabilitation determination unit may include: a sensor interface unit that communicates with the first sensor and the second sensor; a controller that generates operation success information when the load measured in the first sensor is the reference load or more, while the angle information through the sensor interface unit from the second sensor is within a reference angle range and generates operation failure information when the load measured in the first sensor is below the reference load, while the angle information through the sensor interface unit from the second sensor is within the reference angle range; and an output unit that outputs the operation success information and the operation failure information.

Still yet another exemplary embodiment of the present invention provides a legs rehabilitation method of a legs rehabilitation device, including: sensing position or motion information of one leg of a rehabilitating patient; sensing load information provided by a support surface of the other leg of the rehabilitating patient; and determining whether the rehabilitation of the rehabilitating patient succeeds, based on the position information and the load information.

The determining whether the rehabilitation succeeds may include determining that the legs rehabilitation fails, when the load information is below a predetermined reference load, while a cursor moving by the position or motion of the leg reaches a target.

The determining whether the rehabilitation succeeds may include determining that the legs rehabilitation succeeds, when the load information is the predetermined reference load or more, while the cursor moving by the position or motion of the leg reaches a target.

The method may further include sensing angle information of one leg.

The determining whether the rehabilitation succeeds may include determining that the legs rehabilitation fails, when the load information is below the predetermined reference load or the angle information is out of the reference angle range, while the cursor moving by the position or motion of the leg reaches a target.

The determining whether the rehabilitation succeeds may include determining that the legs rehabilitation succeeds, when the load information is the predetermined reference load or more and the angle information is within the reference angle range, while cursor moving by the position or motion of the leg reaches a target.

### BRIEF DESCRIPTION OF THE DRAWINGS

FIG. 1 is a diagram showing a legs rehabilitation device according to an exemplary embodiment of the present invention;

FIG. 2 is a diagram showing a rehabilitation determination unit shown in FIG. 1;

FIG. 3 is a diagram showing a legs rehabilitation device implemented using position or motion information according to the exemplary embodiment of the present invention;

FIG. 4 is a diagram showing a legs rehabilitation operation performed by a rehabilitating patient according to the exemplary embodiment of the present invention;

FIG. 5 is a diagram showing a tilting sensor as a second sensor;

FIG. 6 is a diagram showing an accelerometer as a second sensor;

FIG. 7 is a diagram showing a magnetic sensor as a second sensor;

FIG. 8 is a flowchart showing a legs rehabilitation operation of a legs rehabilitation device shown in FIG. 1;

FIG. 9 is a diagram showing implementation of a legs rehabilitation operation using the legs rehabilitation device according to the exemplary embodiment of the present invention;

FIG. 10 is a diagram showing a display screen of an output unit according to application of a game function to a legs rehabilitation device of FIG. 9;

FIG. 11 is a diagram showing implementation of are habilitation operation using the legs rehabilitation device according to another embodiment of the present invention;

FIG. 12 is diagram showing a skeleton model of a patient formed using a depth image acquired by a capture device of the legs rehabilitation device shown FIG. 11;

FIG. 13 is diagram showing a structure where the rehabilitation device according to the exemplary embodiment is connected with a terminal having other game functions in order to allow a patient to perform games, together with other users; and

FIG. 14 is a diagram a structure where the rehabilitation device according to the exemplary embodiment is connected with other rehabilitation devices in order to allow a patient to perform games, together with other patients.

### DETAILED DESCRIPTION

Various advantages and features of the present invention and methods accomplishing thereof will become apparent from the following description of embodiments with reference to the accompanying drawings. The present invention may, however, be embodied in many different forms without being limited to the embodiments set forth herein. However, the exemplary embodiments of the present invention will be described in detail so that a person with ordinary skilled in the art to which the present invention pertains may easily perform the technical ideas of the present invention.

In the drawings, the exemplary embodiments of the present invention are not limited to a specific form shown and are exaggerated in order to obtain clarity. Further, like reference numerals denote like components throughout the specification.

The exemplary embodiment of present invention provides a legs rehabilitation device for improving the weight support capability and balance capability of legs having a degraded function and a legs rehabilitation method using the same.

Generally, legs serve to support the weight of a body at the time of upright or walking. However, legs of a patient that is paralyzed or of which muscular strength weakens may not normally perform the role of supporting the body.

Therefore, the legs rehabilitation device according to the exemplary embodiment of the present invention may be used for rehabilitation of a leg with degraded support capability among both legs. The patient with both legs having the degraded function may alternately use the rehabilitation device. The rehabilitating patient may perform the rehabilitation through training of moving one leg in a state in which the other leg with a degraded function due to paralysis, weakening of muscular strength or the like, supports a weight through the rehabilitation device proposed in the exemplary embodiment of the present invention.

Therefore, the legs rehabilitation device of the exemplary embodiment of the present invention may perform the training of changing or moving one leg to several positions while the change in load from the other leg with a degraded function is measured. In this case, the legs rehabilitation device may perform the rehabilitation of the rehabilitating patient by determining whether a load above a predetermined reference is applied to one leg.

That is, when the load of one leg with a degraded function is below a reference load, it is determined that the training of improving the support capability of the rehabilitating patient fails. Unlike this, when the load of one leg with a degraded function is above a reference load, it is determined that the training of improving the support capability of the rehabilitating patient succeeds.

In addition, the legs rehabilitation device may further measure an angle (for example, a degree where a knee is bent) of a joint part or an angle of a portion of a leg according to the operation of a joint of one leg. In this case, a joint may be at least one of a knee joint, a hip joint, and an ankle joint. The portion of the leg may be a thigh and a lower leg. It can be appreciated that the rehabilitation is successfully performed through whether an angle (or bending} of one leg is included within a predetermined reference angle range in a state in which the load above a predetermined reference is applied from one leg.

In the case where the angle of the leg is further measured, when the angle of the leg is included within the predetermined reference angle in the state in which the load of one leg with the degraded function is above the predetermined load, it may be determined that the training of improving the support capability of the rehabilitating patient succeeds. In this case, when the load of one leg is below the reference load or the angle of the leg is deviates from the reference range, it may be determined that the training of improving the support capability of the rehabilitating patient fails.

The training of improving support capability of one leg may be performed according to the operation of the other leg by changing or moving the position of the other leg, during the determination whether the rehabilitation training succeeds through the change in a load of one leg (and/or the change in an angle of one leg).

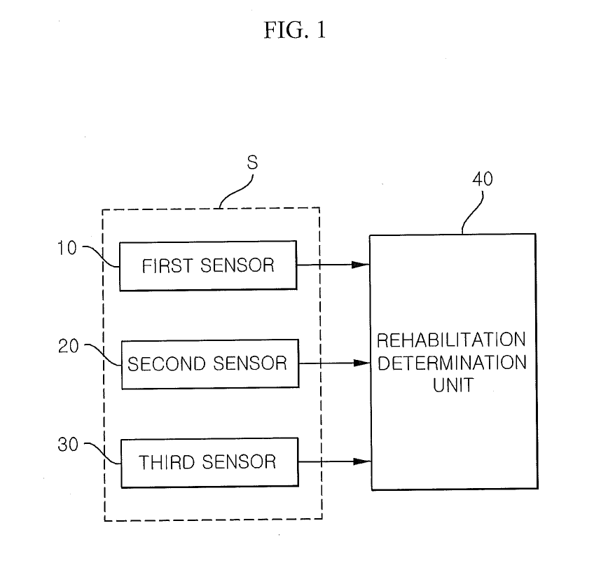

The legs rehabilitation device implemented to have the above-mentioned function will be described with reference to FIGS. 1 to 8.

FIG. 1 is a diagram showing the legs rehabilitation device according to the exemplary embodiment of the present invention.

Referring to FIG. 1, the legs rehabilitation device includes a first sensor 10, a second sensor 20, a third sensor 30, and a rehabilitation determination unit 40.

The first sensor 10 is a sensor that measures a load applied from a support surface of one leg of a rehabilitating patient. The first sensor 10 may sense the load generated by the change in support force of one leg. The first sensor 10 generates load information provided through the support surface of one leg. In this configuration, one leg generating the load information through the first sensor 10 is a leg with a degraded function and is a leg that is the rehabilitation object in order to improve the support force.

The second sensor 20 is a sensor that measure a joint angle (or bending) or an angle of a portion of one leg due to an exercise (contraction or relaxation) of a muscle of one leg of the rehabilitating patient. In this case, the joint includes at least one of a knee joint, a hip joint, and an ankle joint. The portion of the leg may be a thigh and a lower leg. The second sensor may be attached to the joint (for example, at least one of a knee joint, a hip joint, and an ankle joint) part of one leg of the rehabilitating patient. The second sensor 20 generates the angle information of the leg according to the muscle exercise of the joint part of one leg of the rehabilitating patient. The second sensor may be a tilting sensor attached to various locations of the leg, in addition to the joint. When the ankle joint, the knee joint, and the hip joint are bent, a shank or a femur is inclined from a vertical state and when the ankle joint, the knee joint, and the hip joint extend, the shank or the femur becomes vertical.

The third sensor 30 is a sensor that senses the position or the operation of the other leg of the rehabilitating patient. The third sensor 30 may be attached to the other leg of the rehabilitating patient. For example, the third sensor 30 may be attached to feet or shoes of the rehabilitating patient. The third sensor 30 generates the positional information according to the operation of the other leg to which the third sensor 30 is attached.

The rehabilitation determination unit 40 may communicate with each of the first sensor 10, the second sensor 20, and the third sensor 30. The rehabilitation determination unit 40 may communicate with the sensors 10, 20, and 30 in a wired or wireless manner. The rehabilitation determination unit 40 may receive the load information from the first sensor 10. The rehabilitation determination unit 40 may receive the angle information from the second sensor 20. The rehabilitation determination unit 40 may receive the position or motion information from the third sensor 30.

The rehabilitation determination unit 40 may receive the position or motion information from the third sensor 30 while the change in the load information from the first sensor 10 is sensed. The rehabilitation determination unit 40 may determine whether the rehabilitation operation succeeds, based on the load information and the position or motion information from the first sensor 10. That is, the load information from the first sensor 10 may be below the reference load. In this case, the rehabilitation determination unit 40 determines that the rehabilitation operation fails. However, the load information from the first sensor 10 is the reference load or more and a cursor moving by the position or motion information from the third sensor 30 may reach the predetermined target. In this case, the rehabilitation determination unit 40 determines that the rehabilitation operation succeeds.

The rehabilitation determination unit 40 may receive the angle information from the second sensor 20 while the change in the load information from the first sensor 10 is sensed. The rehabilitation determination unit 40 may determine whether the rehabilitation operation succeeds, based on the load information and the angle information from the first sensor 10. That is, the load information from the first sensor 10 may be below the reference load. In this case, the rehabilitation determination unit 40 determines that the rehabilitation operation fails. However, the load information from the first sensor 10 may be the reference load or more and the angle information from the second sensor 20 may be within the reference angle range. In this case, the rehabilitation determination unit 40 determines that the rehabilitation operation succeeds.

Further, the rehabilitation determination unit 40 may receive the angle information from the second sensor 20 and the position or motion information from the third sensor 30 while the change in the load information from the first sensor 10 is sensed. The rehabilitation determination unit 40 may determine whether the rehabilitation operation succeeds, based on the load information from the first sensor 10, the angle information from the second sensor 20, and the position or motion information from the third sensor 30. That is, the load information from the first sensor 10 may be below the reference load, the angle information from the second sensor 20 may be out of the reference angle range, and/or the cursor moving by the position or motion of the third sensor 30 may not reach the target. In this case, the rehabilitation determination unit 40 determines that the rehabilitation operation fails. However, the load information from the first sensor 10 may be the reference load or more and the angle information from the second sensor 20 may be within the reference angle range. In this case, the rehabilitation determination unit 40 determines that the rehabilitation operation succeeds, when the cursor moving by the position or motion of the third sensor 30 reaches the target.

The rehabilitation determination unit 40 may output whether the legs rehabilitation operation succeeds to the rehabilitating patient. As a result, the rehabilitation determination unit 40 may feedback information for legs rehabilitation to the rehabilitating patient (or, a doctor, a nurse, or the like).

Therefore, the rehabilitation device 40 according to the exemplary embodiment of the present invention uses at least one of the angle information from the second sensor 20 and the position or motion information from the third sensor 30 and the load information from the first sensor 10 to determine the success or failure of the rehabilitation, such that the rehabilitation may be provided to the rehabilitating patient.

For example, it is assumed that the rehabilitating patient performs the rehabilitation operation so as to improve the support capability of the left leg.

The left leg of the rehabilitating patient is positioned on the support surface (for example, a foothold) of the first sensor 10. The second sensor 20 is attached to the left leg of the rehabilitating patient. In this case, the third sensor 30 is attached to the right leg of the rehabilitating patient.

The rehabilitating patient supports his/her weight using the left leg. In this case, the support force (load) is applied through the sole of the left leg and the support surface of the first sensor 10. Therefore, the first sensor 10 senses the load information, for example, the load that is generated according to the support force.

In addition, the second sensor 20 is attached to the left leg of the rehabilitating patient.

The second sensor 20 senses the information on the joint angle according to the contraction and relaxation of the muscle around the joint or the angle (bending) of the body part.

The third sensor 30 outputs the information according to the position or motion thereof according to the change in the position or the motion of the leg in at least one of six directions of front and rear-left and right-up and down directions of the right leg. The rehabilitation determination unit 40 may receive the load information from the first sensor 10 and the angle information from the second sensor 20 even while the position or motion information is received from the third sensor 30. In this case, when the weight load of the left leg of the rehabilitating patient is reduced, the rehabilitation determination unit 40 may confirm through the load information output from the first sensor 10 that the load is reduced. In addition, the rehabilitation determination unit 40 may determine through the angle information output from the second sensor 20 that the extension of the leg is appropriate.

The rehabilitation determination unit 40 determines whether the load below the minimum load to be held by the rehabilitating patient is applied from the first sensor 10. It may be confirmed from the determination of the rehabilitation determination unit 40 whether the legs rehabilitation operation of the rehabilitating patient succeeds.

On the other hand, when the rehabilitation operation is performed so as to improve the support capability of the right leg, the right leg of the rehabilitating patient is positioned on the support surface (for example, a foothold) of the first sensor 10. The second sensor 20 is attached to the right leg of the rehabilitating patient. In this case, the third sensor 30 is attached to the left leg of the rehabilitating patient.

In the exemplary embodiment of the present invention, the second sensor 20 may be optionally used. The rehabilitation device may include or may not include the second sensor 20.

The rehabilitating patient may perform the rehabilitation through the training of improving the support capability of the legs by using the legs rehabilitation device proposed in the exemplary embodiment of the present invention.

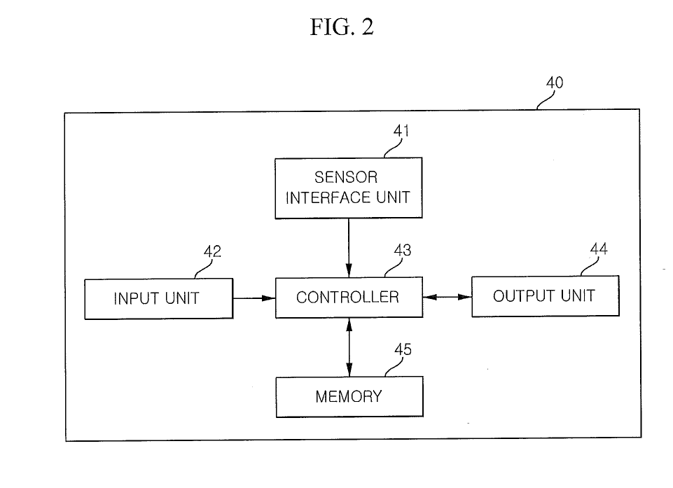

FIG. 2 is a diagram showing the rehabilitation determination unit shown in FIG. 1.

Referring to FIG. 2, the rehabilitation determination unit 40 includes a sensor interface unit 41, an input unit 42, a controller 43, an output unit 44, and a memory 45.

The sensor interface unit 41 may communicate with the first sensor 10 and receive the load information from the first sensor 10. In this case, the load information from the first sensor 10 is information according to the load sensing of the sensor. The sensor interface unit 41 may communicate with the second sensor 20 and receive the angle information from the second sensor 20. In this case, the angle information from the second sensor 20 is information obtained by sensing the angle of the leg bent according to the contraction or relaxation of the muscle around the joint. The sensor interface unit 41 may communicate with the third sensor 30 and receive the position or motion information from the third sensor 30.

Meanwhile, the sensor interface unit 41 may communicate with each of the first sensor 10, the second sensor 20, and the third sensor 30 in a wired or wireless manner.

The input unit 42 may receive the control signal for controlling the operation of the rehabilitation determination unit 40 or the rehabilitating patient information, or the like, from the user. For example, the rehabilitating patient information includes information on a name, an age, a height, a weight, a rehabilitation record, or the like. The input unit 42 may be implemented through a keyboard, a mouse, a touch pen, a touch pad, or the like.

The controller 43 receives through the sensor interface unit 41 the load information from the first sensor 10, the angle information from the second sensor 20, and the position and motion information from the third sensor 30.

The controller 43 compares the load information from the first sensor 10 with the reference load. The controller 43 generates the operation failure information when the load information from the first sensor 10 is below the reference load.

The controller 43 determines whether the angle information from the second sensor 20 is within the reference angle range. The controller 43 generates the operation failure information when the angle information from the second sensor 20 is out of the reference angle range.

In addition, the controller 43 determines whether the cursor moving by the position or motion information from the third sensor 30 reaches a target. The controller 43 generates the operation failure information when the cursor moving by the position or motion information from the third sensor does not reach a target.

Meanwhile, the controller 43 may determine that the load information from the first sensor 10 is the reference load or more and the angle information from the second sensor 20 is the reference angle range or more. In this case, the controller 43 generates the operation success information when the cursor moving by the position or motion information from the third sensor 30 reaches a target.

That is, the controller 43 generates the operation success information when the controller 43 determines that the load information from the first sensor 10 is the reference load or more and the angle information from the second sensor 20 is the reference angle or more, while the cursor moving by the position or motion information of the other leg through the position or motion information received through the third sensor 30 reaches a target.

When the sensing information sensed from each sensor 10, 20, and 30 satisfies all the reference values (for example, reference load, reference angle range, target, or the like), the controller 43 may output the operation success information.

The controller 43 may store the rehabilitating patient information input through the input unit 42, or the like, in the memory 45, together with the operation failure information or the operation success information. In addition, the controller 43 may count a try frequency, a success frequency, or the like when each rehabilitating patient is subjected to the rehabilitation and may store the counted information in the memory 45.

In this case, the controller 43 may output the information related to the rehabilitation operation through the output unit 44.

The output unit 44 outputs the operation failure information generated in the controller 43. In addition, the operation success information may be output. The output unit 44 may include a display device, a speaker, or the like.

The memory 45 may store a driving program for operating the rehabilitation determination unit 40. The memory 45 may store the target information corresponding to the position or motion information from the third sensor 30. In addition, when a game function is in connection with the rehabilitation determination unit 40, the memory 45 may store game programs.

The memory 45 may store various pieces of information (patient information, patient treatment record (for example, try frequency, success frequency), or the like) related to the treatment of the rehabilitating patient and may additionally store various pieces of information in addition to the above-mentioned information.

The above-mentioned rehabilitation determination unit 40 according to the exemplary embodiment of the present invention may feedback the rehabilitation operation state to the rehabilitating patient so as to improve the leg support capability of the rehabilitating patient.

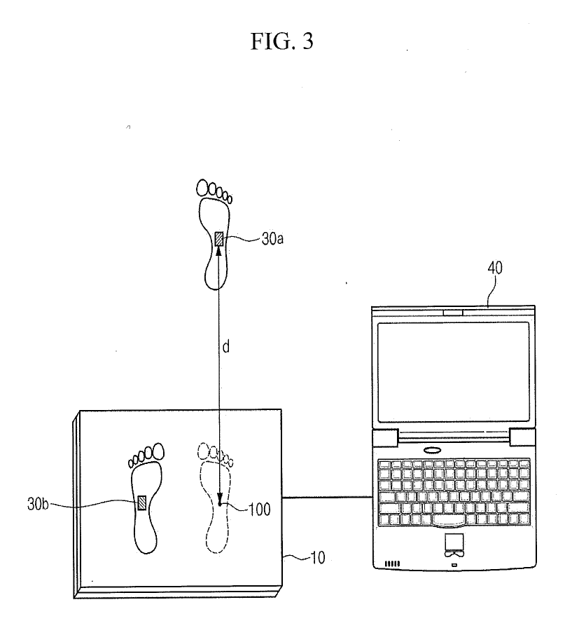

FIG. 3 shows the legs rehabilitation device implemented by using the position and motion information according to the exemplary embodiment of the present invention.

Referring to FIG. 3, the legs rehabilitation device includes the first sensor 10, third sensors 30a and 30b, and the rehabilitation determination unit 40.

The first sensor 10 measures, for example, the load applied from the support surface of the left leg or the right leg. The first sensor 10 has the support surface (a foothold) on which the rehabilitating patient is positioned. The first sensor 10 outputs the measured load information to the rehabilitation determination unit 40.

The third sensor 30a is attached to the right leg (for example, a right foot) so as to measure the operation of the right leg of the rehabilitating patient. The third sensor 30b may be attached to the left leg (for example, a left foot) so as to measure the operation of the left leg of the rehabilitating patient.

Presently, the right leg to which the third sensor 30a is attached is operated and the third sensor 30a outputs the position or motion information according to the right leg.

For example, the third sensor 30a may measure a distance d based on the position of the reference point 40 determined on the first sensor 10. The third sensor 30a may sense the operation through the distance change with the first sensor 10.

The third sensor 30a measures the distance change with the first sensor 10 to output the positional information of the leg to which the third sensor 30a is attached to the rehabilitation determination unit 40.

The third sensor 30a outputs the motion information of the leg to which the third sensor 30a is attached to the rehabilitation determination unit 40.

In this case, the operation of training the support force of the left leg of the rehabilitating patient is shown. In this case, the position or motion information received from the third sensor 30a attached to the right leg may be received. In this case, the rehabilitating patient may perform the training of improving the support capability of the left leg while the right leg is operated.

The rehabilitation determination unit 40 receives the sensing information (for example, load information and position or motion information) sensed from the first sensor 10 and the third sensor 30a. The rehabilitation determination unit 40 determines whether the load information from the first sensor 10 is the reference load or more, while the position or motion information from the third sensor 30a is changed. Alternatively, the rehabilitation determination unit 40 determines whether the load information from the first sensor 10 is the reference load or more, while the cursor moving by the position or motion information from the third sensor 30a reaches a target.

When the load information from the first sensor 10 is the reference load or more, the rehabilitation determination unit 40 determines that the rehabilitation operation succeeds. However, when the load information from the first sensor 10 is below the reference load, the rehabilitation determination unit 40 determines that the rehabilitation operation fails.

Meanwhile, the rehabilitation determination unit 40 may further include the second sensor 20 and additionally determines whether the angle information from the second sensor 20 is within the reference angle range to determine whether the rehabilitation operation succeeds.

When the second sensor 20 is used, the rehabilitation determination unit 40 may provide the rehabilitating patient with the information so as to appropriately contract or relax the joint of one leg, in the state in which the rehabilitating patient maintains a constant load applied to one leg. In addition, the rehabilitation determination unit 40 outputs the operation success information when the cursor moving by the motion or position of the other leg reaches a target, thereby inducing the treatment operation of the rehabilitating patient.

In addition, the rehabilitation determination unit 40 additionally determines whether the positional information or motion information of the other leg from the third sensor 30a is received for the predetermined time, thereby determining that the operation succeeds. Further, when the rehabilitation determination unit 40 is operated at the operation speed of the other leg above the predetermined reference speed, it may determine from the information of the third sensor 30a that the operation fails. Therefore, the efficiency of the rehabilitation operation may be improved by slowly moving the other leg for the rehabilitating patient.

The rehabilitation determination unit 40 may output the rehabilitation operation failure information through the display device, the speaker, or the like, to the rehabilitating patient when the rehabilitation operation fails. As a result, the rehabilitating patient corrects his/her rehabilitation operation, thereby improving the efficiency of the rehabilitation operation.

That is, the rehabilitating patient may operate the right left while constantly maintaining the weight load to the left leg so as not to receive the operation failure. Therefore, the support capability of the left leg of the rehabilitating patient may be improved.

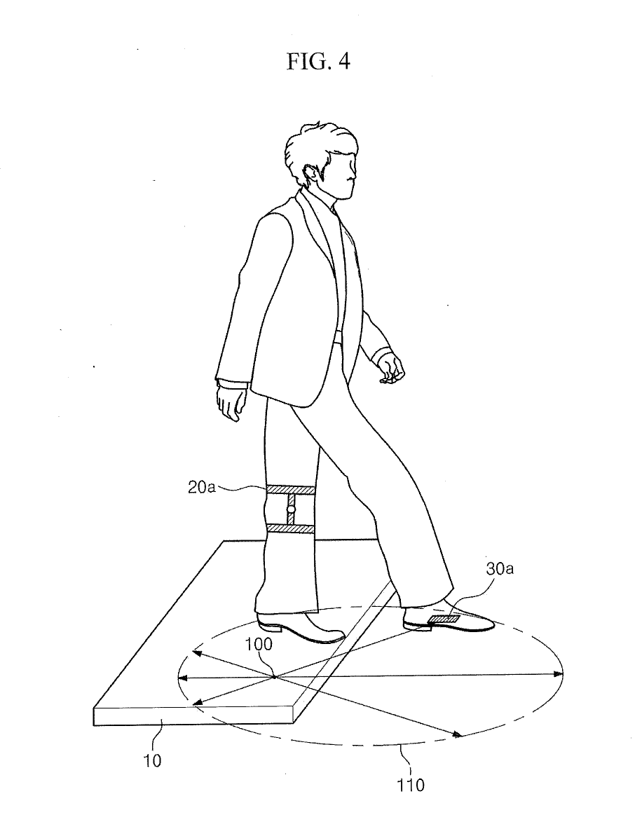

FIG. 4 shows the legs rehabilitation operation performed by the rehabilitating patient according to the exemplary embodiment of the present invention.

Referring to FIG. 4, the first sensor 10 includes the support surface at which the rehabilitating patient is positioned. The first sensor 10 measures the load applied from the support surface of the first sensor 10.

The first sensor 10 may include the load sensor that measures the load of the left leg of the patient.

A second sensor 20a may be attached to the leg of the rehabilitating patient. The second sensor 20a is attached to the joint (for example, a knee joint) of the left leg of the rehabilitating patient and the third sensor 30a is attached to the right leg. Only one of the second sensor 20a and the third sensor 30a may be attached according to the rehabilitation part of the rehabilitating patient.

The second sensor 20a may include angle measurement sensors that measure the angle information. The second sensor 20a may include an electric angle meter, a variable resistance angle meter and a motion capture angle meter.

For example, the second sensor 20a generates the angle information formed at the leg according to the contraction or relaxation of the muscle around the joint.

As necessary, the second sensor 20a may be omitted and a legs rehabilitation device may be constituted by only the first sensor 10 and the third sensor 30a.

The third sensor 30a may include operation measurement sensors that measure the motion information.

For example, the third sensor 30a may generate the positional information including a distance moving from the reference point, based on the reference point 40 determined on the first sensor 10. In this case, the moving area 110 of the third sensor 30a is shown based on the reference point 100.

As another example, the third sensor 30a generates the motion information in the front and rear and the left and right directions. The first sensor 10 is a load sensor that senses whether the weight load having a predetermined size or more is maintained at the one leg. The second sensor 20 is an angle measurement sensor that determines whether the muscle around the joint of one leg is appropriately contracted and relaxed. The third sensor 30a is a sensor that senses the position or motion according to the predetermined operation performance for rehabilitation.

For example, the third sensor 30a may be a plurality of touch sensors that are installed at a predetermined position. In this case, each of the plurality of touch sensors may store the positional information from the touch sensor therein and provide the positional information to the rehabilitation determination unit 40.

Meanwhile, the third sensor 30a may generate another information that shows the leg operation, not the positional information, for example, the operational information such as the acceleration information, or the like.

In this case, the third sensor 30a may be implemented by the acceleration sensor, or the like, and may provide the acceleration information according to the leg operation to the rehabilitation determination unit 40.

As described above, the first sensor 10, the second sensor 20a, and the third sensor 30a of the present invention may be implemented as various sensors, in addition to the above-mentioned sensors.

The second sensor may be a sensor according to a scheme of indirectly measuring a joint angle of a lower limb. As the second sensor, a tilting sensor, a gyro sensor, an accelerometer, a magnetic sensor, an ultrasonic distance measuring device, and the like, may be used.

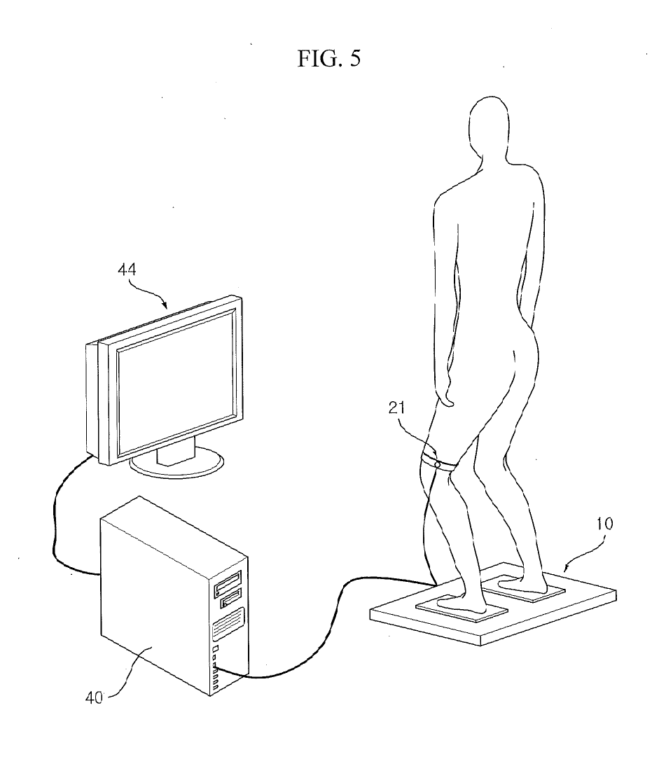

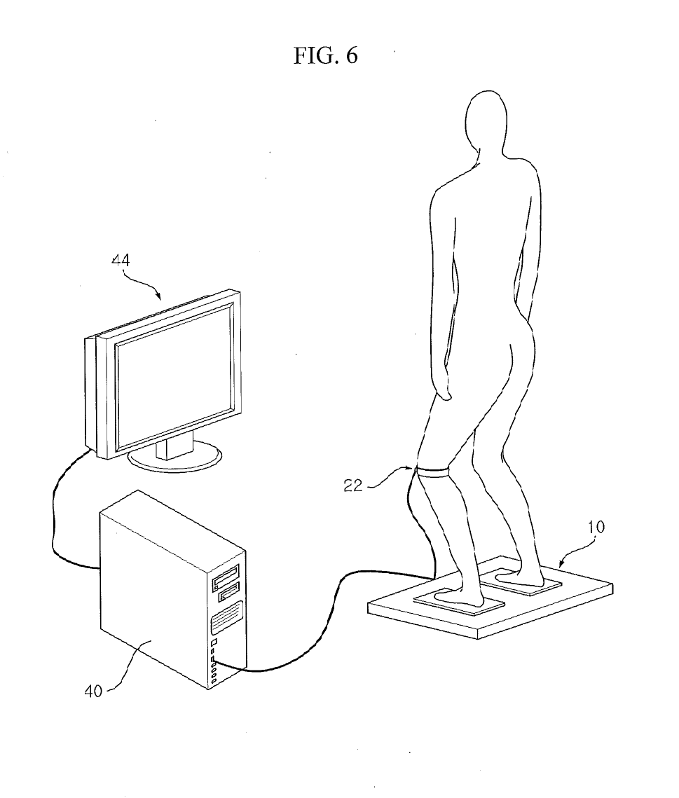

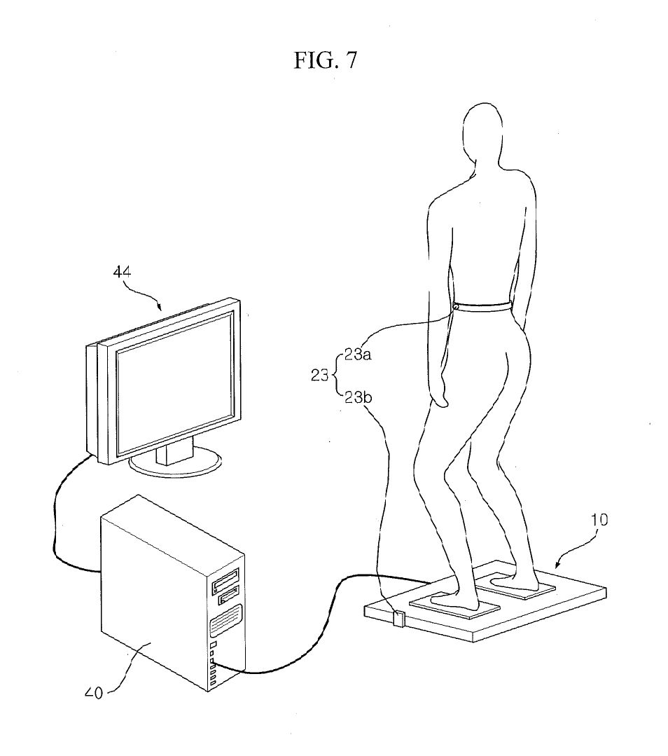

FIG. 5 illustrates a tilting sensor used as a second sensor, FIG. 6 illustrates an accelerometer used as the second sensor, and FIG. 7 illustrates a magnetic sensor as the second sensor.

Referring to FIG. 5, the joint angle of the lower limb may be indirectly measured by measuring the slope of the thigh or the lower leg using the tilting sensor 21 or the gyro sensor. For this, the tilting sensor 21 or the gyro sensor may be attached to one region of the thigh or the lower leg. When a user bends or straightens a joint of the lower limb in a state where the tilting sensor 21 or the gyro sensor is attached to the thigh or the lower leg, the slope of the attachment region may be measured by the tilting sensor 21 and the gyro sensor and the joint angle of the lower limb may be calculated using the measurement value.

Referring to FIG. 6, the joint angle of the lower limb may be indirectly measured by attaching the accelerometer 22 to one region of the lower limb and measuring the acceleration of the lower limb. The accelerometer may be attached to various regions of the lower limb, however, preferably to the front of a knee joint. The knee joint moves forward when the lower limb is bent, and moves backward when the lower limb is straightened. Therefore, the joint angle of the lower limb may be indirectly measured by measuring the acceleration of the knee joint using the accelerometer 22.

Also, in a state where feet of the user touch the floor, if the user bends the lower limb, a body of the user downwardly moves, and if the user straightens the lower limb, the body of the user upwardly moves. Therefore, the joint angle of the lower limb may be indirectly measured by measuring a distance between a reference point and a predetermined region of the body of the user using the magnetic sensor and the ultrasonic distance measuring device.

Specifically, referring to FIG. 7, to indirectly measure the angle of the joint using the magnetic sensor, a permanent magnet 23a is attached to the predetermined region of the user body, for example, a waist and a magnetic sensor 23b is disposed at a floor. Since a distance between the permanent magnet and the magnetic sensor becomes close when bending the corresponding lower limb, the magnetic force increases. On the contrary, when straightening legs, the distance between the magnet and the magnetic sensor becomes distant, the magnetic force decreases. It is possible to measure the absolute distance between the magnetic sensor and the permanent magnet using a change in the magnitude of the magnetic force, or to measure the relative movement between the magnetic sensor and the permanent magnet.

Even when using the ultrasonic distance measuring device as the second sensor, the joint angle of the lower limb may be indirectly measured in a similar manner. The user bends or straightens the lower limb in a state where an ultrasonic generator is installed on the floor and an ultrasonic reflector is attached to a predetermined region of the user, for example, a waist, a distance between the ultrasonic generator and the ultrasonic reflector becomes close or distant. Therefore, a change in the joint angle of the lower limb may be indirectly measured by measuring the distance that is measured by the ultrasonic distance measuring device based on the change in the joint angle of the lower limb of the user.

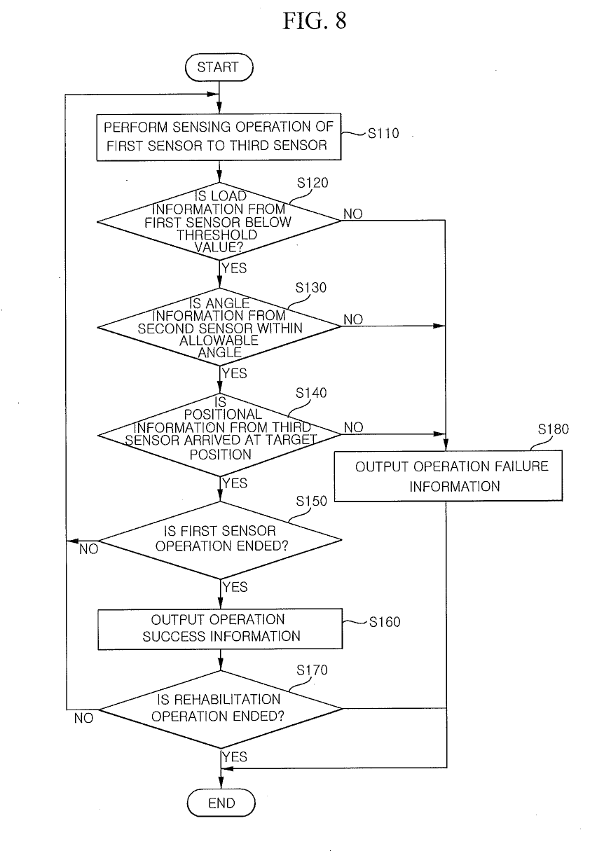

FIG. 8 is a flow chart showing the legs rehabilitation operation of the legs rehabilitation device shown in FIG. 1.

Referring to FIG. 8, at step S110, the first sensor 10, the second sensor 20, and the third sensor 30 each performs the sensing operation. The first sensor 10 is a sensor which the rehabilitating patient may step up and senses the load applied through the support surface with the other leg. The second sensor 20 senses the angle of the leg. The third sensor 30 is attached to one leg of the rehabilitating patient and senses the operation of one leg. The first sensor 10, the second sensor 20, and the third sensor 30 output the sensing information, for example, the load information to the rehabilitation determination unit 40.

At step S120, the rehabilitation determination unit 40 determines whether the load information received from the first sensor 10 is below the threshold value (for example, threshold load), while the operation of the third sensor 30 is sensed. That is, the rehabilitation determination unit 40 determines whether the load received from the first sensor 10 is below the reference load.

At step S120, if it is determined that the load information from the first sensor 10 is below the reference load (threshold value), the rehabilitation determination unit 40 proceeds to step S180.

At step S180, the rehabilitation determination unit 40 outputs the operation failure information and ends the rehabilitation operation. Alternatively, the rehabilitation determination unit 40 may initialize the rehabilitation operation.

At step S120, if it is determined that the load information from the first sensor 10 is not below the reference load (that is, reference load or more), the rehabilitation determination unit 40 proceeds to step S130.

At step S130, the rehabilitation determination unit 40 determines whether the sensing information received from the second sensor 20, that is, the angle information is within the predetermined reference angle range, while the operation of the third sensor 30 is sensed.

At step S130, if it is determined that the angle information from the second sensor 20 is not within the reference angle range, the rehabilitation determination unit 40 proceeds to step S180.

At step S130, if it is determined that the angle information from the second sensor 20 is within the reference angle range, the rehabilitation determination unit 40 proceeds to step S140.

At step S140, the rehabilitation determination unit 40 determines whether the cursor moving by the position or motion information from the third sensor 30 reaches a target. At step S140, the rehabilitation determination unit 40 may determine from the third sensor 30 whether the cursor is positioned at the target for the predetermined time.

At step S140, the rehabilitation determination unit 40 may determine based on the position or motion information from the third sensor 30 whether the cursor is operated within the predetermined speed range (in order to determine as the failure the case whether the cursor is rapider than the predetermined speed or slower than the predetermined speed). The determination operation at steps S120 and S130 is continuously performed in the rehabilitation determination unit 40 while the cursor moving by the position or motion information at step S140 reaches a target. Therefore, the rehabilitation determination unit 40 may proceed to step S180 if the rehabilitation determination unit 40 does not satisfy the conditions of steps S120 and S130 while the cursor moving by the position or motion information reaches a target at step 140.

Meanwhile, steps S120 to S140 is for the convenience of explanation and thus, may be performed in a different order or may be performed simultaneously. Even in this case, for example, when the second sensor 130 is not used, step S130 may be omitted.

At step S140, the rehabilitation determination unit 40 proceeds to step S180 if the cursor moving by the position or motion information from the third sensor 30 does not reach a target.

At step S140, the rehabilitation determination unit 40 proceeds to step S150 if the cursor moving by the position or motion information from the third sensor 30 reaches a target.

At step S150, the rehabilitation determination unit 40 determines whether the operation of the third sensor 30 ends. At step S150, when the operation of the third sensor 30 does not end, the rehabilitation determination unit 40 proceeds to step S110.

At step S150, when the operation of the third sensor 30 ends, the rehabilitation determination unit 40 proceeds to step S160.

The rehabilitation determination unit 40 outputs the operation success information at step S160 and proceeds to step S170.

At step S170, the rehabilitation determination unit 40 confirms whether the rehabilitation operation ends. When the rehabilitation operation does not end, the rehabilitation determination unit 40 proceeds to step S110 to receive the sensing information from the first sensor 10, the second sensor 20, and the third sensor 30.

When the rehabilitation operation ends, the rehabilitation determination unit 40 ends the rehabilitation operation.

The exemplary embodiment of the present invention proposes the legs rehabilitation device so as to improve the support capability of the leg with the degraded function, which does not normally function, among two legs. The legs rehabilitation device according to the exemplary embodiment of the present invention includes the sensors (the first sensor 10 and the second sensor 20) that measures the weight load of one leg that is paralyzed or of which muscular strength weakens and the angle of the leg and the sensor (the third sensor 30) that senses the operation of the other leg that is not paralyzed. The legs rehabilitation device according to the exemplary embodiment of the present invention uses the information received through two sensors (the first sensor 10 and the third sensor 30) or three sensors (the first sensor 10, the second sensor 20, and the third sensor 30) to feedback to the user whether the problem is successfully performed, thereby improving the support capability of the paralyzed leg.

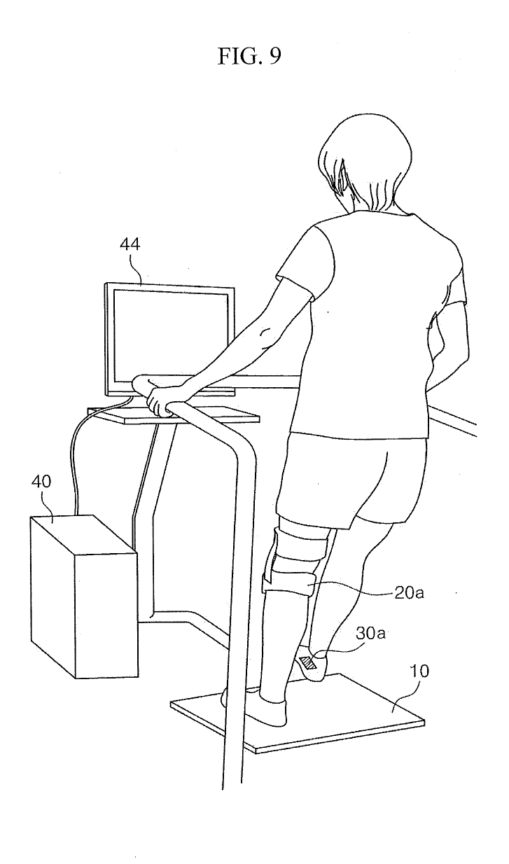

FIG. 9 shows the implementation of the rehabilitation operation using the legs rehabilitation device according to the exemplary embodiment of the present invention.

Referring to FIG. 9, the legs rehabilitation device to which the game function is applied may be used for the legs rehabilitation.

The legs rehabilitation device includes the first sensor 10, the second sensor 20a, the third sensor 30a, and the rehabilitation determination unit 40.

The first sensor 10 measures the load applied through the right leg of the rehabilitating patient. The first sensor 10 outputs the load information.

The second sensor 20a is attached to the right leg of the rehabilitating patient and measures the angle formed by the knee joint according to the motion of the knee joint part. The second sensor 20a outputs the angle information.

The third sensor 30a is attached to the left leg of the rehabilitating patient and senses the operation according to the motion of the left leg. The third sensor 30a outputs the position or motion information.

The rehabilitation determination unit 40 may include the game function so as to improve the efficiency of the rehabilitation operation of the rehabilitating patient. In this case, the rehabilitation determination unit 40 includes an output unit 44 implemented as the display type.

Therefore, the rehabilitation determination unit 40 uses the sensing information received through the first sensor 10, the second sensor 20a, and the third sensor 30a to generate the game data, thereby implementing the game function. The rehabilitation determination unit 40 uses the sensing information received through each sensor (the first sensor 10, the second sensor 20a, and the third sensor 30a) to operate the game.

The rehabilitation determination unit 40 provides the user with the information to appropriately maintain the appropriate extension motion (or bending) of the supported leg, in the state in which the predetermined load is applied to the leg supporting the body of the rehabilitating patient. In this case, the rehabilitation determination unit 40 leads to various motions of the other leg to provide the rehabilitation (for example, improving the support capability of the left leg) to the rehabilitating patient.

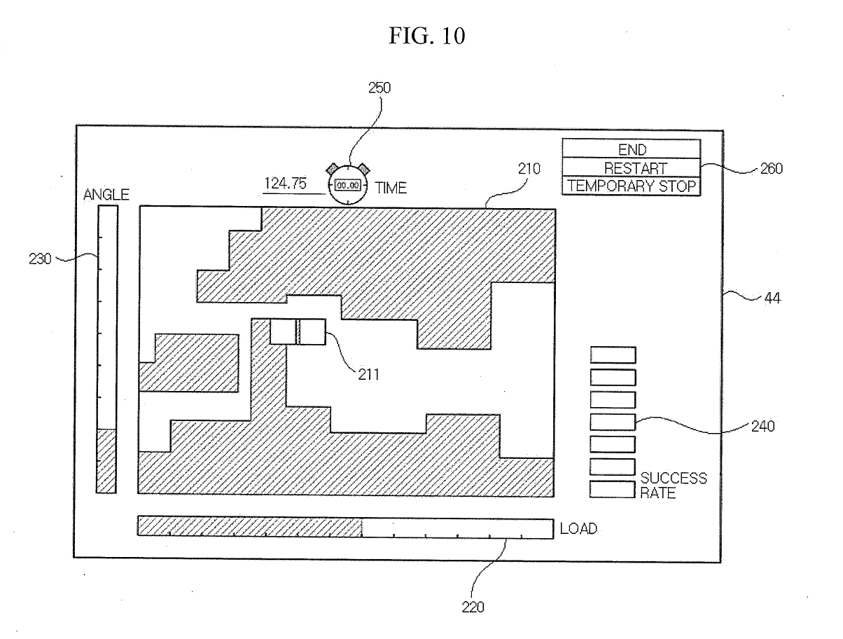

FIG. 10 shows display screen of the output unit according to the application of the game function to the legs rehabilitation device of FIG. 9.

Referring to FIG. 10, the display screen 44 of the output unit outputs the image data corresponding to the game data generated by the rehabilitation determination unit 40 according to the game function implementation.

It is assumed that for example, a board cleaner game is applied to the display screen 44. The display screen 44 includes a board area 210, a load information display area 220, an angle information display area 230, a success probability display area 240, a timer display area 250, and a function selection area 260.

It is assumed that the board cleaner game function implemented through the display screen 44 is associated with the rehabilitation operation shown in FIG. 6.

The board area 210 includes a board cleaner 211. In this case, the board cleaner 211 may move within the board area according to the operation of the sensor positioned at, for example, the right foot. When the operation according to the conditions of the rehabilitation operation is successfully performed, the display colors of the board area 210 in which the board cleaner 211 moves may be changed (displaying the shape such as the shape in which the board is erased. In order to operate the board cleaner 211, the rehabilitation determination unit 40 may use the positional information received from the third sensor 30a.

The load information display area 220 may display the load information applied through one leg of the rehabilitating patient, for example, the left leg. In order to display the load information in the load information display area 220, the rehabilitation determination unit 40 may use the load information received from the first sensor 10.

The angle information display area 230 may display the angle information applied through one leg of the rehabilitating patient, for example, the knee joint of the left leg. In order to display the angle information in the load information display area 220, the rehabilitation determination unit 40 may use the angle information received from the second sensor 20a.

The success probability display area 240 is an area that indicates a ratio of the erased board region to the overall board area in the state in which the sensing information received from the first sensor 10, the second sensor 20a, and the third sensor 30a generated by the rehabilitating patient is satisfied. Alternatively, the success probability display area 240 may indicate a success frequency to a overall try frequency according to the rehabilitation operation.

The timer display area 250 is an area that indicates information on an elapsed time from the time when the board cleaner game starts.

The function selection area 260 displays various key buttons that controls the game function. In this case, the function selection area 260 may include the key buttons that controls various functions such as ending, restarting, temporary stopping, or the like, according to the game function implementation from the rehabilitating patient, or the like. The function selection area 260 may be implemented in a touch screen type. Input signals may be generated from the rehabilitating patient through the function selection area 260 implemented in the touch screen type.

Therefore, the rehabilitating patient may have an interest in the rehabilitation training through the game function implemented in the rehabilitation device. Further, the rehabilitating patient may improve the efficiency of the rehabilitation training through the rehabilitation device implementing the game function.

For example, the rehabilitation device according to the exemplary embodiment of the present invention may be applied to the rehabilitation of the patient with paralyzed legs due to stroke, traumatic brain injury, or the like.

This may use various games such as fruit harvesting in addition to the board cleaner game.

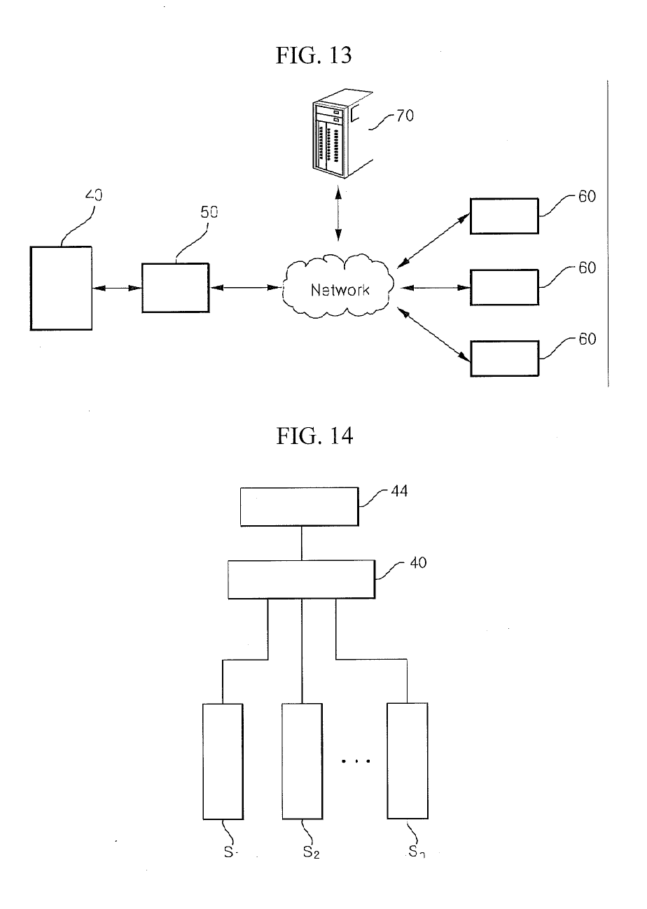

Referring to FIG. 13, the game function implemented by the legs rehabilitation device according to the exemplary embodiment of the present invention may be performed alone but may be performed simultaneously with multiple users in a single game space through the network. To this end, the legs rehabilitation device according to the exemplary embodiment of the present invention may include the communication interface 50. The communication interface 50 accesses the legs rehabilitation device according to the exemplary embodiment of the present invention to the server 70 or the terminal 60 of another user through the network so as to perform data communication. The patient uses the communication interface 50 to simultaneously perform the game through the network, together with multiple users. The network may be, for example, an internal network or Internet, etc. The terminal 60 of another user accessing the network may be a device having the same configuration as the legs rehabilitation device according to the exemplary embodiment of the present invention. However, if the terminal 60 of another user accesses the legs rehabilitation device of the server 70 or the patient by compatible protocol to perform the same games as games implemented by the legs rehabilitation device according to the exemplary embodiment of the present invention, the terminal 60 does not need to have the same configuration as the leg rehabilitation system according to the exemplary embodiment of the present invention and may be, for example, a game only terminal.

In addition, referring to FIG. 14, multiple patients use the legs rehabilitation device according to the exemplary embodiment of the present invention without passing through a separate server or network to perform the rehabilitation training while simultaneously performing the game in the same game space. Sensors S are each provided to the multiple patients and each sensor S is connected with the rehabilitation determination unit 40. The rehabilitation determination unit 40 generates the game data corresponding to each patient, based on the signal transferred from the plurality of sensors S. The game data for each patient generated by the rehabilitation determination unit 40 is displayed as the image by the output unit 44. In the exemplary embodiment of the present invention, the single rehabilitation determination unit 40 is configured to process the signals transferred from the plurality of sensors S but if necessary, but the rehabilitation determination unit 40 may be provided in plural so as to individually process the signals from each sensor S, if necessary.

Meanwhile, the lower limb angle of the patient may be by various schemes.

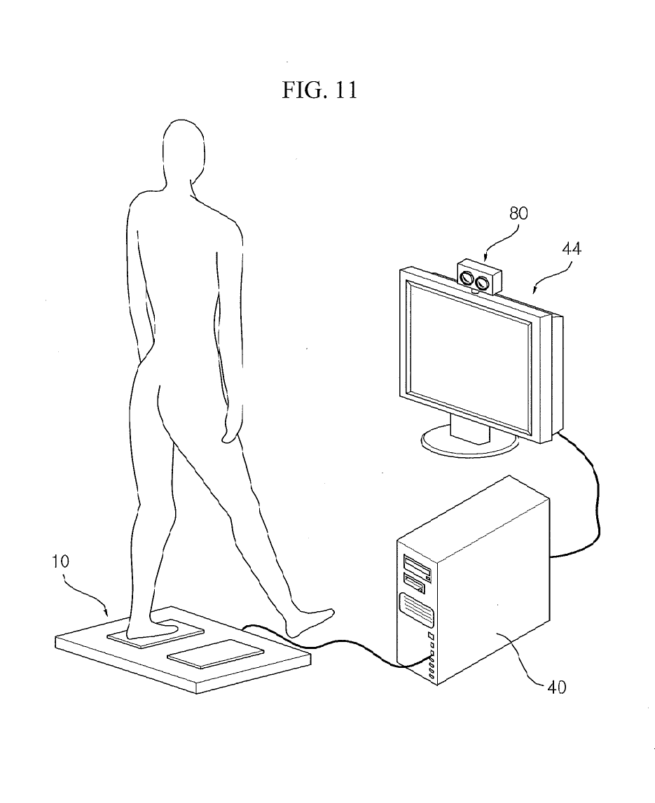

FIG. 11 is a diagram showing implementation of an operation of a rehabilitation using the legs rehabilitation device according to another exemplary embodiment of the present invention.

The legs rehabilitation device according to another embodiment of the present invention includes a first sensor 10, a capture device 80, and a rehabilitation determination unit 40, and an output unit 44.

In the legs rehabilitation device according to the exemplary embodiment, the measurement of the joint angle of one leg of the rehabilitating patient and the sensing of the position or operation of the other leg of the rehabilitating patient are implemented by the capture device 80. That is, the capture device 80 of the legs rehabilitation device according to the exemplary embodiment integrally implements the functions of the second sensor and the third sensor of the legs rehabilitation device according to the embodiment shown in FIG. 1.

The first sensor 10 measures the load applied through the right leg of the rehabilitating patient. The first sensor 10 outputs the load information.

The capture device 80 according to the embodiment of the present invention is used to visually monitor the leg of the patient. The capture device 80 is configured to capture the depth image of the patient. The capture device 80 may be configured to capture video with depth information via any suitable technique e.g., time-of-flight, structured light, stereo image, etc. As such, capture device 80 may include a depth camera, a video camera, stereo cameras, and/or other suitable capture devices. In addition, the capture device 80 may include at least two physically separated cameras monitoring the patients from various angles so as to acquire a visual stereo data. The depth image may include the plurality of observed pixels and each observed pixel has the observed depth value. The observed depth value includes the depth information of the patient observed from the capture device 80.

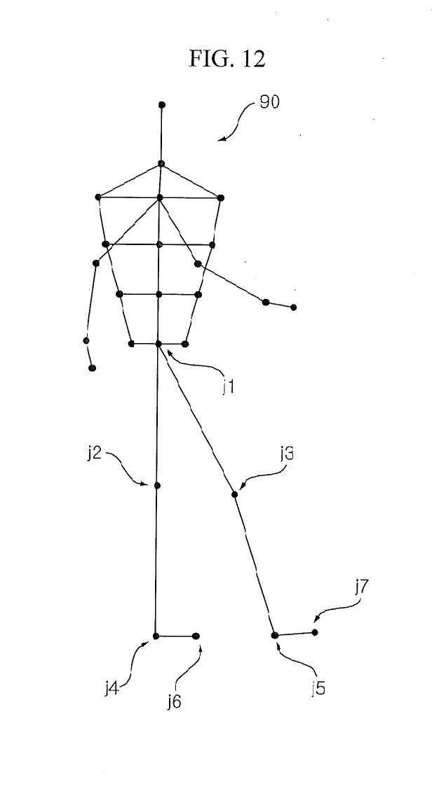

The rehabilitation determination unit 40 may be configured to receive the depth image from the capture device 80 to display the leg of the patient as a model. FIG. 12 is diagram showing a skeleton model of a patient formed using a depth image acquired by a capture device of the legs rehabilitation device shown FIG. 11. The model including at least two body portions may include at least one joint j1-j7. Each joint allows at least one body portion to move with respect to at least one other body portion. In addition, each body portion of the model may include at least one structural member (that is, bones) and the joints are positioned at intersecting portions of the adjacent bones. Some bones may correspond to the bones of the patients in dissection and other bones may not have the corresponding bones of the patients in dissection.

Bones and joints may collectively configure a skeleton model. The skeleton model may be a component of the model. The skeleton model may include a joint between at least one skeleton member and the adjacent skeleton members with respect to the respective body portions.

When the patient performs rehabilitation training, the depth image of the patient body including both legs of the patient is captured by the capture device 80. The rehabilitation determination unit 40 forms the skeleton model 90 of the patient using the captured depth image by the capture device 80. In addition, the rehabilitation determination unit 40 analyzes the formed skeleton model 90 of the patient to sense the angle of one leg and the position or operation of the other leg of the patient.

What is claimed is:

1. A legs rehabilitation device comprising:
a first sensor that senses a load applied from a support surface of one leg of a rehabilitating patient;
a second sensor that senses an angle of the one leg;
a third sensor that senses position or motion of the other leg of the rehabilitating patient; and
a rehabilitation determination unit for communicating with the first sensor, the second sensor, and the third sensor, the rehabilitation determination unit determining whether legs rehabilitation succeeds, based on load information received from the first sensor, angle information received from the second sensor, and position or motion information received from the third sensor.

2. The device of claim 1 wherein the rehabilitation determination unit determines that the rehabilitation fails when at least one of cursors moving in connection with the load information received through the first sensor, the angle information received through the second sensor, and the position or motion information of the other leg received through the third sensor is out of a predetermined range.

3. The device of claim 1 wherein the first sensor is a load sensor that senses the load applied from one leg through the support surface.

4. The device of claim 1 wherein the second sensor is an angle sensor that is attached around a joint part of one leg of the rehabilitating patient to measure an angle of a joint part.

5. The device of claim 1 wherein the third sensor is a positional sensor that is attached to the other leg of the rehabilitating patient to sense the position of the other leg.

6. The device of claim 5 wherein the positional sensor is an operation measurement sensor that outputs the distance information based on a predetermined reference point.

7. The device of claim 5 wherein the positional sensor is a touch sensor that receives the positional information by contacting one of a plurality of touch sensors having the predetermined positional information.

8. The device of claim 1 wherein the rehabilitation determination unit provides a game function to the rehabilitating patient, based on the load information received through the first sensor, the angle information received through the second sensor, and the position and motion information received through the third sensor.

9. The device of claim 1 wherein the second sensor includes at least one of a tilting sensor and a gyro sensor attached to a slope or a lower leg or a thigh of one leg of the rehabilitating patient, an accelerometer attached to a knee joint, and a magnetic sensor or an ultrasonic distance measuring device measuring a distance between a reference point and a specific body portion of the rehabilitating patient, and a joint angle of the one leg is indirectly measured.

10. The device of claim 1 wherein the second sensor and the third sensor are a capture device capturing a depth image of the rehabilitating patient.

11. The device of claim 10 wherein the rehabilitation determination unit receives the depth image from the capture device to form a model of the patient.

12. The device of claim 11 wherein the rehabilitation determination unit analyzes the model to calculate an angle of one leg of the patient and calculate a position of the other leg of the patient.

13. The device of claim 10 wherein the capture device includes a depth camera.

14. A legs rehabilitation device comprising:
a first sensor that senses a load applied from a support surface of one leg of a rehabilitating patient;
a second sensor that senses position or motion of the other leg of the rehabilitating patient; and
a rehabilitation determination unit for communicating with the first sensor and the second sensor, the rehabilitation determination unit and determining whether legs rehabilitation succeeds, based on load information received from the first sensor and the position and motion information received from the second sensor.

15. The device of claim 14 wherein the rehabilitation determination unit determines that the rehabilitation fails when at least one of cursors moving in connection with the load information received through the first sensor and the position or motion information of the other leg received through the second sensor is out of a predetermined range.

16. A legs rehabilitation method of a legs rehabilitation device comprising:
sensing position or motion information of one leg of a rehabilitating patient;
sensing load information provided by a support surface of the other leg of the rehabilitating patient; and
determining whether rehabilitation of the rehabilitating patient succeeds based on the position or motion information and the load information.

17. The method of claim 16 wherein the determining whether the rehabilitation succeeds includes determining that the legs rehabilitation fails when at least one of cursors moving in connection with the load information and the position or motion information of the one leg is out of a predetermined range.

18. The method of claim 16 further comprising sensing angle information of one leg.

19. A legs rehabilitation device comprising:
a first sensor that senses a load applied from a support surface of one leg of a rehabilitating patient;
a second sensor that senses an angle of a joint part of the one leg;
a third sensor that senses position or motion of the other leg of the rehabilitating patient;
a rehabilitation determination unit for communicating with the first sensor, the second sensor, and the third sensor, the rehabilitation determination unit generating game data based on load information received from the first sensor, angle information received from the second sensor, and position or motion information received from the third sensor;
an output unit that outputs image data corresponding to the game data generated by the rehabilitation determination unit; and
a communication interface that shares the game data generated by the rehabilitation determination unit with at least one terminal of other users through a network to allow a patient to perform a game simultaneously with other users.

20. The device of claim 19 wherein the legs rehabilitation device is directly connected with the terminals of other users.

21. The device of claim 19 wherein the legs rehabilitation device is connected with the terminals of other users through a server.

22. A legs rehabilitation device comprising:
a first sensor assembly that includes a first sensor sensing a load applied from a support surface of one leg of a first rehabilitating patient, a second sensor sensing an angle of the one leg of the first rehabilitating patient, and a third sensor sensing position or motion of the other leg of the first rehabilitating patient;
a second sensor assembly that includes a fourth sensor sensing a load applied from a support surface of one leg of a second rehabilitating patient, a fifth sensor sensing an angle of a joint part of one leg of the second rehabilitating patient, and a sixth sensor sensing the position or motion of the other leg of the second rehabilitating patient, wherein the first rehabilitating patient uses the first sensor assembly and the second rehabilitating patient uses the second sensor assembly;
a rehabilitation determination unit for communicating with the first sensor, the second sensor, the third sensor, the fourth sensor, the fifth sensor, and the sixth sensor, the rehabilitation determination unit generating game data based on load information received from the first sensor and the fourth sensor, angle information received from the second sensor and the fifth sensor, and position or motion information received from the third sensor and the sixth sensor; and
an output unit that outputs image data corresponding to the game data generated by the rehabilitation determination unit.

23. The device of claim 22 wherein the rehabilitation determination unit includes:
a sensor interface unit that communicates with the first sensor, the second sensor, the third sensor, the fourth sensor, the fifth sensor, and the sixth sensor;
a controller that generates operation success information at the game when the load measured in the first sensor is the reference load or more and the angle information measured in the second sensor is within the reference angle range and generates operation failure information at the game when the load measured in the first sensor is below the reference load or the angle information measured in the second sensor is out of the reference angle range, while the cursor moving by the position or motion information through the sensor interface unit from the first sensor reaches a target; and
an output unit that displays the game operation success and the game operation failure.
